(19) United States
(12) Patent Application Publication
(10) Pub. No.: US 2019/0083122 A1
(43) Pub. Date: Mar. 21, 2019

(54) MORCELLATOR WITH ROTATING FEEDERS

(71) Applicant: COVIDIEN LP, Mansfield, MA (US)

(72) Inventors: SCOTT J. PRIOR, SHELTON, CT (US); NIKOLAI D. BEGG, WAYLAND, MA (US)

(21) Appl. No.: 16/115,070

(22) Filed: Aug. 28, 2018

Related U.S. Application Data
(60) Provisional application No. 62/561,289, filed on Sep. 21, 2017.

(51) Int. Cl. A61B 17/32; A61B 17/34

(52) U.S. Cl. CPC A61B 17/32002; A61B 17/00234; A61B 2017/320024; A61B 17/3423

(57) ABSTRACT

A morcellator includes an elongated shaft and first and second feeders. The elongated shaft defines a longitudinal axis and has a proximal portion and a distal portion. The elongated shaft defines a channel along the longitudinal axis between the proximal and distal portions. The first feeder is disposed in distal portion of the elongated shaft and extends along and is rotatable about a first rotation axis. The second feeder is disposed in the distal portion of the elongated shaft, and extends along and is rotatable about a second rotation axis that is parallel to and offset from the first rotation axis. The first and second feeders are configured to cooperate to draw tissue disposed between the first and second feeders proximally into the channel.

### CROSS REFERENCE TO RELATED APPLICATION

The present application claims the benefit of and priority to U.S. Provisional Application Ser. No. 62/561,289, filed on Sep. 21, 2017 the entire contents of which are incorporated herein by reference.

### BACKGROUND

### 1. Technical Field

The present disclosure relates to tissue morcellation and, more specifically, to tissue morcellators which can be used for partial or total removal of body tissue from a body cavity.

### 2. Discussion of Related Art

In minimally invasive surgical procedures, operations are carried out within the body by using elongated instruments inserted through small entrance openings in the body. The initial opening in the body to allow passage of instruments to the interior of the body may be a natural passageway of the body, or it can be created by a tissue-piercing instrument such as a trocar, or by a small incision into which a cannula is inserted.

Minimally invasive procedures may be used for partial or total removal of body tissue from a body cavity, e.g. partial or total hysterectomy, myomectomy, nephrectomy, cholecystectomy, lobectomy, and other procedures including thoracic, abdominal, laparoscopic, and endoscopic procedures. During such procedures, it is common that a cyst, fibroid, myoma, tumor, or other affected tissue needs to be removed via the access opening or through a cannula. Various types of entrapment devices have been disclosed to facilitate this procedure. In many procedures where cancerous tumors are removed, removal of the specimen in an enclosed environment, e.g., a specimen bag, is highly desirable to inhibit seeding of cancer cells (i.e., portions of cancer cells contacting healthy tissue).

Several minimally invasive surgical procedures require the bulk removal of body tissue through a limited surgical opening. As such, the tissue needs to be morcellated within the body cavity into smaller pieces of tissue to facilitate removal.

There is a continuing need for morcellators for bulk removal of tissue.

### SUMMARY

In accordance with aspects of the present disclosure, a morcellator is provided including an elongated shaft and first and second feeders. The elongated shaft defines a longitudinal axis and has a proximal portion and a distal portion. The elongated shaft defines a channel along the longitudinal axis between the proximal and distal portions. The first feeder is disposed in distal portion of the elongated shaft, extends along a first rotation axis, and is rotatable about the first rotation axis. The second feeder is disposed in the distal portion of the elongated shaft, extends along as second rotation axis, and is rotatable about the second rotation axis. The second rotation axis is parallel to and offset from the first rotation axis. The first and second feeders are configured to cooperate to draw tissue disposed between the first and second feeders proximally into the channel.

In an aspect of the present disclosure, the first and second rotation axes are perpendicular to the longitudinal axis.

In another aspect of the present disclosure, the first and second feeders are cylindrical.

In another aspect of the present disclosure, the first feeder has a first segment and a second segment. The first and second segments define a passage therebetween in communication with the channel.

In still another aspect of the present disclosure, the first feeder includes teeth extending radially outward from an outer surface thereof. Edges of the teeth may be sharpened. Additionally or alternatively, teeth of first feeder may be arranged in rows parallel to the first rotation axis and/or adjacent rows of teeth of the first feeder may be offset along the first rotation axis from one another.

In yet another aspect of the present disclosure, the second feeder includes teeth extending radially outward from an outer surface of the second feeder. In such aspects, the teeth of the first and second feeders may be arranged in rows with the rows of the second feeder offset from the rows of the first feeder.

In still yet another aspect of the present disclosure, the first and second feeders are conical.

In another aspect of the present disclosure, the first and second rotation axes are parallel to and offset from the longitudinal axis.

In yet another aspect of the present disclosure, the first feeder has an apex and a base and is oriented with the apex proximal of the base and disposed along the first rotation axis. The base defines a plane orthogonal to the first rotation axis. The plane defined by the base of the first feeder may be positioned distal of a distal end of the distal portion of the elongated shaft while the apex of the first feeder is positioned within the distal portion of the elongated shaft.

In still another aspect of the present disclosure, the morcellator is a side loaded morcellator.

In another aspect of the present disclosure, the proximal portion of the elongated shaft is configured to releasably couple to a handle.

A morcellation system provided in accordance with aspects of the present disclosure includes a handle having a motor and a morcellator including any of the features of the morcellator detailed above or the morcellators detailed herein. The first and second feeders of the morcellator are operably coupled to the motor and configured to rotate in response to activation of motor such that the first and second feeders cooperate to draw tissue disposed between the first and second feeders proximally into the channel.

A method of removing tissue from a surgical site through a small opening is also provided in accordance with the present disclosure and includes positioning a distal portion of a morcellator adjacent tissue to be removed from a surgical site, contacting first and second rotating feeders of a morcellator with the tissue to be removed, and rotating the first and second rotating feeders such that the tissue is drawn proximally between the first and second rotating feeders and into a channel of the morcellator.

In aspects of the present disclosure, contacting the first and second rotating feeders includes passing a tool through the distal portion of the morcellator to grasp the tissue to be removed and drawing the tissue proximally into contact with the first and second rotating feeders.

Further, to the extent consistent, any of the aspects described herein may be used in conjunction with any or all of the other aspects described herein.

### BRIEF DESCRIPTION OF THE DRAWINGS

Various aspects of the present disclosure are described hereinbelow with reference to the drawings, which are incorporated in and constitute a part of this specification, wherein:

FIG. 1 is a perspective view of a morcellator provided in accordance with the present disclosure;

FIG. 2 is an enlarged view of a distal portion of the morcellator of FIG. 1;

FIG. 3 is an enlarged view of the indicated area of detail of FIG. 1 with a tenaculum engaged with tissue;

FIG. 4 is a perspective view of the distal portion of the morcellator of FIG. 1 with the tenaculum drawing tissue into the morcellator;

FIG. 5 is a perspective view of a distal portion of another morcellator provided in accordance with the present disclosure;

FIG. 6 is a perspective view of a distal portion of yet another morcellator provided in accordance with the present disclosure;

FIG. 7 is a perspective view of a distal portion of still another morcellator provided in accordance with the present disclosure; and

FIG. 8 is a side view of the distal portion of the morcellator of FIG. 8 with portions shown transparent to illustrate internal components.

### DETAILED DESCRIPTION

Embodiments of the present disclosure are described in detail with reference to the drawings in which like reference numerals designate identical or corresponding elements in each of the several views. As used herein, the term “clinician” refers to a doctor, a nurse, or any other care provider and may include support personnel. Throughout this description, the term “proximal” refers to the portion of the device or component thereof that is closer to the clinician and the term “distal” refers to the portion of the device or component thereof that is further from the clinician.

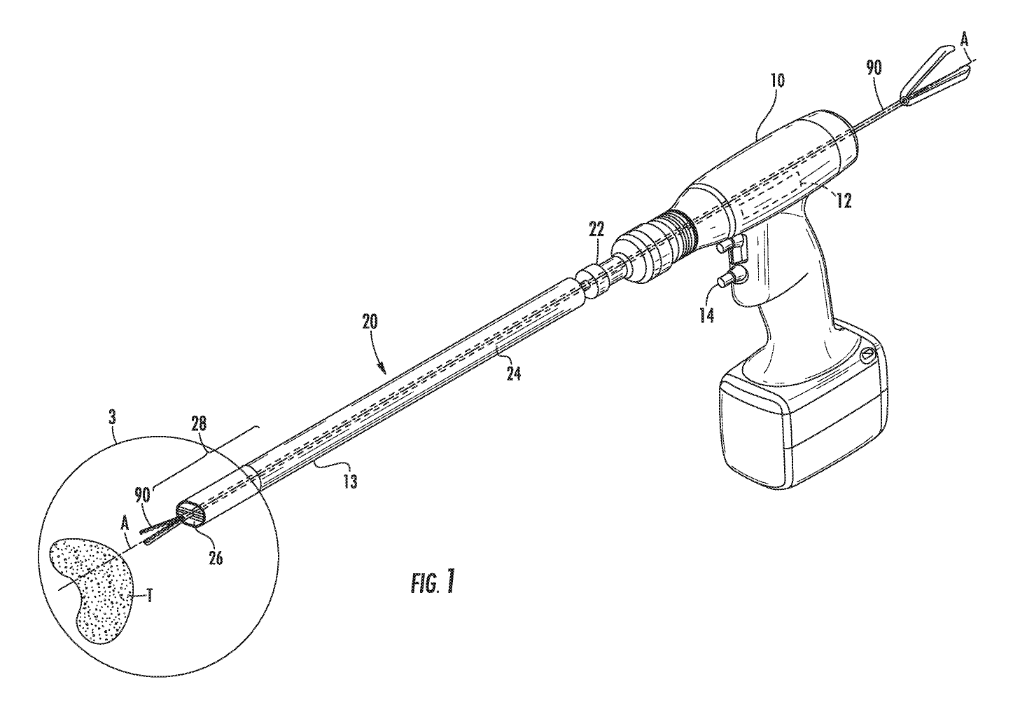

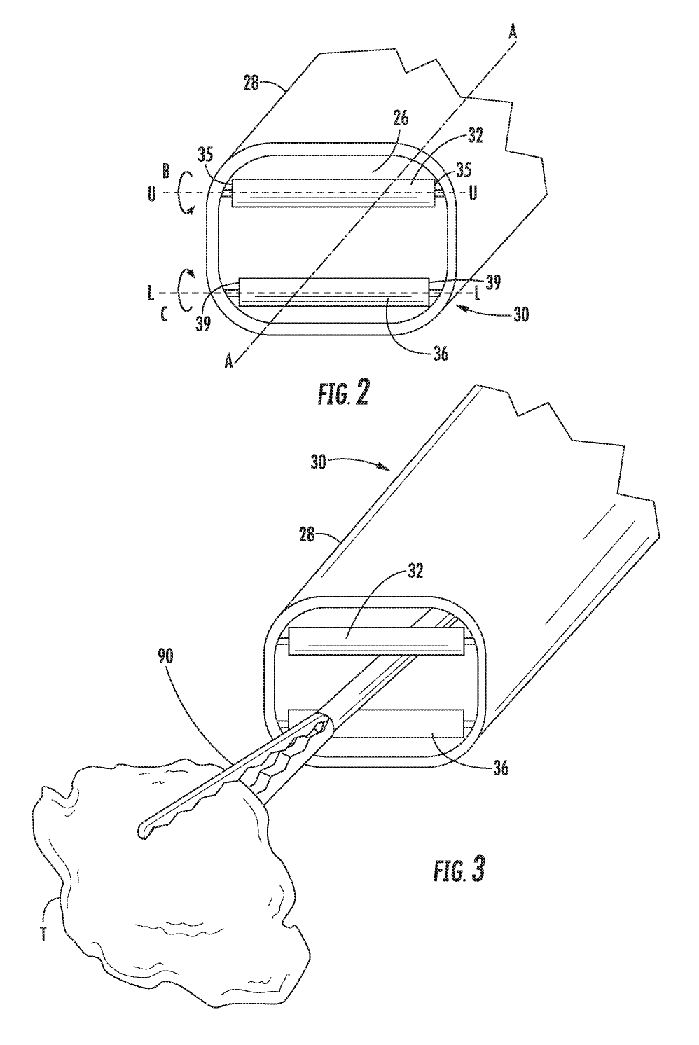

Referring now to FIGS. 1 and 2, a morcellation system is provided in accordance with the present disclosure. The morcellation system includes a handle 10 and a morcellator 20. The morcellator 20 is releasably coupled to the handle 10. The handle 10 includes a driver or motor 12 that is activatable to actuate and/or manipulate the morcellator 20. The handle 10 includes a control interface 14 having one or more controls to activate and/or manipulate the morcellator 20. The motor 12 may be activatable to rotate the morcellator 20 about a longitudinal axis A-A thereof. In addition, the motor 12 may be activatable to actuate the morcellator 20 as detailed below. For a detailed description of exemplary powered or manual handles reference is made to U.S. Patent Publication No. 2015/0141869 and U.S. Pat. No. 9,603,624, the entire contents of each of these disclosures are hereby incorporated herein by reference.

The morcellator 20 includes an elongated shaft 24 that has a proximal portion 22 which is releasably coupled to the handle 10. The elongated shaft 24 extends in a direction along the longitudinal axis A-A of the morcellator 20 and defines a channel 26 between the proximal portion 22 and a distal portion 28 of the morcellator 20. The channel 26 extends through the entire morcellator 20. The morcellator 20 also includes a feeding mechanism 30 (FIG. 2) disposed at least partially within the distal portion 28 of the morcellator 20. The distal portion 28 may be substantially oval in cross-section such that the distal portion 28 is shaped to receive the feeding mechanism 30. The elongate shaft 24 may be substantially oval in cross-section along the entire length thereof or the elongate shaft 24 may be circular in cross-section in the proximal portion 22 and transition to an oval cross-section in the distal portion 28.

With particular reference to FIG. 2, the feeder mechanism 30 includes a first or upper feeder 32 and a second or lower feeder 36. The feeders 32, 36 may be configured as cylinders or in any other suitable configuration. The upper feeder 32 defines and is rotatable about a first or upper rotation axis U-U and the lower feeder 36 defines and is rotatable about a second or lower rotation axis L-L. The upper rotation axis U-U and the lower rotation axis L-L are parallel to one another and are each perpendicular to the longitudinal axis A-A of the morcellator 20. The upper and lower feeders 32, 36 are operably coupled to the motor 12 such that when the motor 12 is energized, the motor rotates the upper feeders 32 about the upper rotation axis U-U in the direction indicated by arrow B and rotates the lower feeders 36 about the lower rotation axis L-L in the direction indicated by arrow C such that the upper and lower feeders 32, 36 are rotated in opposite directions. More specifically, each of the upper and lower feeders 32, 36 rotate inwardly towards the longitudinal axis A-A to feed tissue between feeders 32, 36 and into the channel 26 as described in detail below. The upper and lower feeders 32, 36 may compress and/or crush tissue as the tissue is feed between the feeders 32, 36.

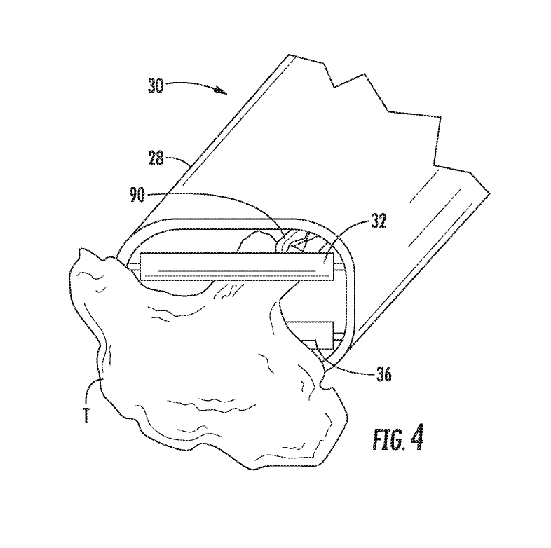

Referring to FIGS. 3 and 4, a method of morcellating tissue T is described in accordance with the present disclosure utilizing the morcellator 20 detailed above. Initially, the distal portion 28 of the morcellator 20 is positioned adjacent the tissue T. With the distal portion 28 positioned adjacent the tissue T, the morcellator 20 is activated to rotate the upper and lower feeders 32, 36 inwardly, as detailed above, to draw the tissue T between the upper and lower feeders 32, 36 and into the channel 26. With the upper and lower feeders 32, 36 rotating inwardly, the morcellator 20 and/or the tissue T is manipulated such that the tissue T is in contact with the upper and lower feeders 32, 36 as shown in FIG. 3. A tool 90, e.g., a tenaculum, may be passed through the channel 26 such that the tool 90 grasps the tissue T to draw the tissue T into contact with the upper and lower feeders 32, 36.

As the tissue T contacts the upper and lower feeders 32, 36, rotation of the upper and lower cylinders 32, 36 draws the tissue T into the channel 26 while compressing the tissue T. The rotation of the upper and lower feeders 32, 36 continues to draw the tissue T into the channel 26 and also pushes the tissue T proximally through the channel 26. The upper and lower feeders 32, 36 have sharpened outside edges 35, 39 (FIG. 2) that cut the tissue T as the tissue T is drawn into the channel 26, although other configurations are also contemplated such as, for example, a sharpened annular distal edge of the distal portion 28 of the morcellator 20. By cutting the tissue T the morcellator 20 is capable of either plunging through the tissue T or skiving segments of the tissue T from the outer surface of the tissue T. Additionally or alternatively, the annular distal edge of the distal portion 28 may include an electrode (not shown) that can be activated with an electrosurgical current to aid in cutting the tissue as the morcellator 20 is plunging or skiving segments of the tissue T from the outer surface of the tissue T. A suitable electrode is disclosed in U.S. patent application Ser. No. 14/325,550, filed Jul. 8, 2014, the entire contents of which are hereby incorporated by reference.

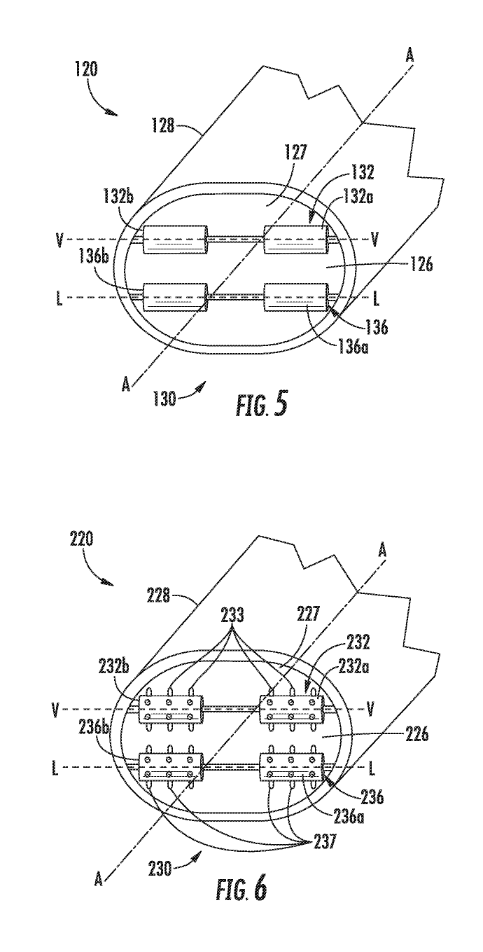

Referring to FIG. 5, a distal portion 128 of another morcellator 120 is provided in accordance with the present disclosure. The morcellator 120 is similar to the morcellator 20 (FIGS. 1-2) with like features represented with a “1” preceding the previous label. As such, for brevity, only the differences will be detailed herein.

The morcellator 120 has a distal portion 128 having a feeder mechanism 130. The feeder mechanism 130 includes an upper feeder 132 having a first segment 132a and a second segment 132b that are coaxial with one another along the upper rotation axis U-U. The morcellator also includes a lower feeder 136 having a first segment 136a and a second segment 136b that are coaxial with one another along the lower rotation axis L-L. The first segments 132a, 136a and the second segments 132b, 136b define a passage 127 therebetween. The passage 127 is in communication with a channel 126 of the morcellator 120 and is sized to permit passage of a tool, e.g., tool 90 (FIG. 1), through the distal portion 128 of the morcellator 120 without contacting the feeders 132, 136. Permitting passage of a tool through the passage 127 without contacting the feeder 132, 136, may extend the life of the feeders 132, 136. In addition, permitting passage of a tool through the passage 127 may reduce resistance to rotation of the feeders 132, 136 which may allow for a reduction in torque required from the motor 12 to rotate the feeders 132, 136 when compared to the feeders 32, 36 detailed above. Further, tissue being drawing through the distal portion 128 of the morcellator 120 may pass through the passage 127 without passing between the feeders 132, 136 which may allow for a reduced force required to rotate the feeders 132, 136.

With reference to FIG. 6, a distal portion 228 of another morcellator 220 is provided in accordance with the present disclosure. The morcellator 220 is similar to the morcellator 120 (FIG. 5) with like features represented with a “2” replacing the “1” as the first number in the previous label. As such, for brevity, only the differences will be detailed herein.

The morcellator 220 has a distal portion 228 having a feeder mechanism 230. The feeder mechanism 230 includes an upper feeder 232 having a first segment 232a and a second segment 232b. Each of the first and second segments 232a, 232b has teeth 233 that extend radially outward from an outer surface of the first and second segments 232a, 232b. The teeth 233 may be arranged in one or more rows about the outer surface of the first and second segments 232a, 232b which may be aligned or offset from teeth 233 of adjacent rows. The teeth 233 may be substantially rectangular in cross-section. As the upper feeder 232 is rotated, the teeth 233 bite into tissue as the tissue is drawn into the channel 226. The edges of the teeth 233 may be sharpened to enhance the biting of tissue. Additionally or alternatively, the teeth 233 may be sharpened and/or serrated to remove or cut off chunks of tissue as the tissue is drawn into the channel 226.

The morcellator 220 also includes a lower feeder 236 having a first segment 236a and a second segment 236b. Each of the first and second segments 236a, 236b has teeth 237 that extend radially outward from an outer surface of the first and second segments 236a, 236b. The teeth 237 are similar to the teeth 233. The teeth 237 may be aligned with the teeth 233 or may be offset from the teeth 233 such that as the upper and lower feeders 232, 236 are rotated, the teeth 233, 237 cooperate to draw tissue into the channel 226.

It is contemplated that the upper and lower feeders 232, 236 may be formed of a single continuous feeder similar to the upper and lower feeders 32, 36 detailed above with respect to morcellator 20 (FIGS. 1-2).

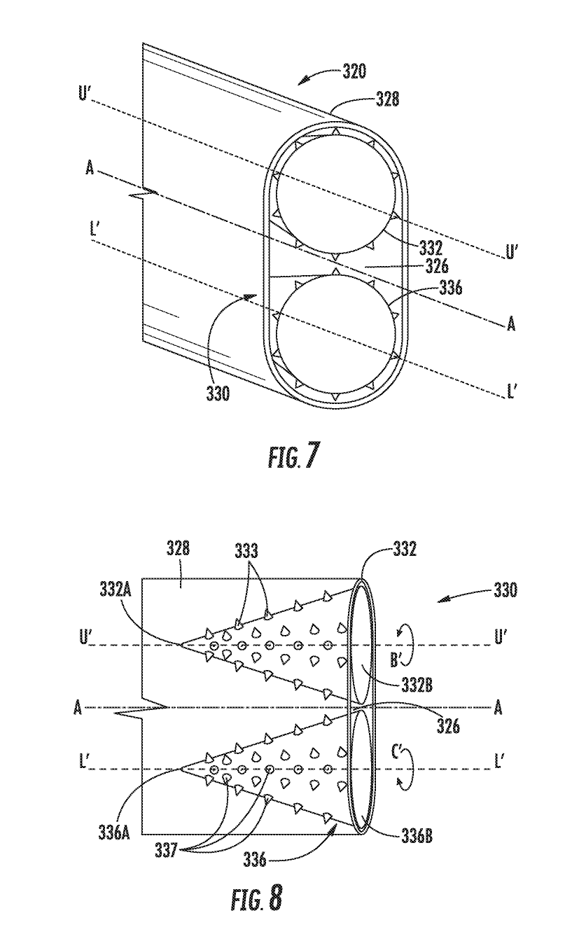

With reference to FIGS. 7 and 8, a distal portion 328 of another morcellator 320 is provided in accordance with the present disclosure. The morcellator 320 is similar to the morcellator 20 (FIGS. 1-2) with like features represented with a “3” preceding the previous label. As such, for brevity, only the differences will be detailed herein. As such, for brevity, only the differences will be detailed herein.

The distal portion 328 of the morcellator 320 includes a feeder mechanism 330 having a first or upper feeder 332 and a second or lower feeder 336. Feeders 332, 336 define cone-shaped configurations, although other configurations are also contemplated. The upper feeder 332 is rotatably disposed along an upper rotation axis U′-U′ that is parallel to and offset from a longitudinal axis A-A of the distal portion 328. The upper feeder 332 has an apex 332a and a circular base 332b. The upper feeder 332 is oriented such that the apex 332a points proximally and the circular base 332b is oriented substantially orthogonal to the upper rotation axis U′-U′. The circular base 332b is positioned distal of a distal end 328a of the distal portion 328, e.g., circular base 332b extends distally from distal end 328a. The lower feeder 336 is rotatably disposed along a lower rotation axis L′-L′ that is parallel to and offset from the upper rotation axis U′-U′ and the longitudinal axis A-A. The lower feeder 336 has an apex 336a and a circular base 336b. The lower feeder 336 is oriented such that the apex 336a points proximally and the circular base 336b is oriented substantially orthogonal to the lower rotation axis L′-L′. The circular base 336b is positioned distal of the distal end 328a of the distal portion 328, e.g., circular base 336b extends distally from distal end 328a. The circular base 332b may be disposed in the same plane as the circular base 336b.

Each of the upper feeder 332 and the lower feeder 336 may include teeth 333, 337, respectively, extending substantially radially outward from outer surfaces of a respective outer surface of the upper and lower feeder 332, 336 which are similar to the teeth 233, 237 detailed above. Thus, the teeth 333, 337 will not be discussed further for reasons of brevity.

As an alternative to the orientation of the upper and lower feeders 332, 336 detailed above, the morcellator 320 may alternatively include cone-shaped feeders 332, 336 arranged similarly as the feeders 32, 36 of morcellator 20 (FIGS. 1-2), with the cone-shaped feeders 332, 336 oriented similarly or oppositely relative to one another. Further still, the morcellator 320 may alternatively include spaced-apart pairs of feeders, similar to those detailed above with respect to the morcellators 120, 220 (FIGS. 5 and 6), and defining cone-shaped configurations with the apexes thereof facing inwardly or oriented in any other suitable configuration.

In use of the morcellator 320, tissue T is brought into contact with outer surfaces of the upper and lower feeders 332, 336 as the upper feeder 332 is rotated in a direction about the upper rotation axis U′-U′ indicated by arrow B′ in FIG. 8 and the lower feeder 336 is rotated in a direction about the lower rotation axis L′-L′ indicated by arrow C′, e.g., the upper and lower feeders 332, 336 are rotated in opposite directions. As tissue contacts the outer surfaces of the upper and lower feeders 332, 336, the tissue is drawn in to the channel 326 and moved proximally through the channel 326. The shape of the upper and lower feeders 332, 336 facilitates urging the tissue proximally as additional tissue is drawn into the upper and lower feeders 332, 336. As will be appreciated from FIG. 8, tissue enters the morcellator 320 offset from the longitudinal axis A-A (via opposed pockets defined between the circular bases 332b, 336b of the upper and lower feeders 332, 336, respectively, and the distal end 328a of the distal end portion 328 of morcellator 320) and is drawn toward the longitudinal axis A-A as the tissue is pulled into the channel 326. Thus, morcellator 320 provides a side-feeding configuration. However, in some embodiments, circular bases 332b, 336b do not extend distally from the distal end 328a of the distal end portion 328 of morcellator 320 and, instead, tissue is received within the space between circular bases 332b, 336b and the annular perimeter of distal end portion 328.

Alternatively or additionally to circular bases 332b, 336b extending distally from the distal end 328a of the distal end portion 328 of morcellator 320, distal end portion 328 may define a cut-out or window (not shown) extending through a side wall thereof to enable side-feeding of tissue into morcellator 320. In this manner, the cone-shaped feeders 332, 336 would pull tissue therethrough similarly as the feeders 32, 36 of morcellator 20 (FIGS. 1-2), except from the side of the distal end portion 328 of morcellator 320. Further, in this configuration, the orientation of cone-shaped feeders 332, 336 helps ensure tissue is urged proximally into and through the distal end portion 328 of morcellator 320, rather than distally therefrom.

While several embodiments of the disclosure have been shown in the drawings, it is not intended that the disclosure be limited thereto, as it is intended that the disclosure be as broad in scope as the art will allow and that the specification be read likewise. Any combination of the above embodiments is also envisioned and is within the scope of the appended claims. Therefore, the above description should not be construed as limiting, but merely as exemplifications of particular embodiments. Those skilled in the art will envision other modifications within the scope of the claims appended hereto.

What is claimed:

1. A morcellator comprising:
an elongated shaft defining a longitudinal axis and having a proximal portion and a distal portion, the elongated shaft defining a channel along the longitudinal axis between the proximal and distal portions;
a first feeder disposed in distal portion of the elongated shaft, the first feeder extending along and rotatable about a first rotation axis; and
a second feeder disposed in the distal portion of the elongated shaft, the second feeder extending along and rotatable about a second rotation axis that is parallel to and offset from the first rotation axis,
wherein the first and second feeders are configured to cooperate to draw tissue disposed between the first and second feeders proximally into the channel.

2. The morcellator according to claim 1, wherein the first and second rotation axes are perpendicular to the longitudinal axis.

3. The morcellator according to claim 1, wherein the first and second feeders are cylindrical.

4. The morcellator according to claim 1, wherein the first feeder has a first segment and a second segment, the first and second segments defining a passage therebetween, the passage in communication with the channel.

5. The morcellator according to claim 1, wherein the first feeder includes teeth extending radially outward from an outer surface of the first feeder.

6. The morcellator according to claim 5, wherein the edges of the teeth of the first feeder are sharpened.

7. The morcellator according to claim 5, wherein the teeth of first feeder are arranged in rows parallel to the first rotation axis.

8. The morcellator according to claim 7, wherein adjacent rows of teeth of the first feeder are offset along the first rotation axis from one another.

9. The morcellator according to claim 7, wherein the second feeder includes teeth extending radially outward from an outer surface of the second feeder.

10. The morcellator according to claim 9, wherein the teeth of the first and second feeders are arranged in rows, the rows of the teeth of the second feeder offset from the rows of the teeth of the first feeder.

11. The morcellator according to claim 1, wherein the first and second feeders are conical.

12. The morcellator according to claim 11, wherein the first and second rotation axes are parallel to and offset from the longitudinal axis.

13. The morcellator according to claim 11, wherein the first feeder has an apex and a base, the first feeder oriented with the apex proximal of the base and disposed along the first rotation axis, the base defining a plane orthogonal to the first rotation axis.

14. The morcellator according to claim 13, wherein the plane defined by the base of the first feeder is positioned distal of a distal end of the distal portion of the elongated shaft and the apex of the first feeder is positioned within the distal portion of the elongated shaft.

15. The morcellator according to claim 1, wherein the morcellator is a side loaded morcellator.

16. The morcellator according to claim 1, wherein the proximal portion of the elongated shaft is configured to releasably couple to a handle.

17. A morcellation system comprising:
a handle having a motor; and
a morcellator including:
an elongated shaft defining a longitudinal axis and having a proximal portion and a distal portion, the elongated shaft defining a channel along the longitudinal axis between the proximal and distal portions, the proximal portion coupled to the handle;
a first feeder disposed in distal portion of the elongated shaft, the first feeder rotatable about and extending along a first rotation axis; and
a second feeder disposed in the distal portion of the elongated shaft, the second feeder extending along and rotatable about a second rotation axis that is parallel to and offset from the first rotation axis,
wherein the first and second feeders are operably coupled to the motor and configured to rotate in response to activation of motor such that the first and second feeders cooperate to draw tissue disposed between the first and second feeders proximally into the channel.

18. The morcellator according to claim 17, wherein the first feeder has a first segment and a second segment, the first and second segments defining a passage therebetween, the passage in communication with the channel.

19. A method of removing tissue from a surgical site through a small opening, the method comprising:
positioning a distal portion of a morcellator adjacent tissue to be removed from a surgical site;
contacting first and second rotating feeders of a morcellator with the tissue to be removed; and
rotating the first and second rotating feeders such that the tissue is drawn proximally between the first and second rotating feeders and into a channel of the morcellator.

20. The method according to claim 9, wherein the contacting the first and second rotating feeders includes passing a tool through the distal portion of the morcellator to grasp the tissue to be removed and to draw the tissue proximally into contact with the first and second rotating feeders.
